(19) United States
(12) Patent Application Publication
(10) Pub. No.: US 2024/0408022 A1
(43) Pub. Date: Dec. 12, 2024

(54) DESIDUSTAT PARTICLES AND COMPOSITIONS THEREOF

(71) Applicant: ZYDUS LIFESCIENCES LIMITED, Gandhinagar, Gujarat (IN)

(72) Inventors: Kumar Kamlesh SINGH, Gandhinagar,Gujarat (IN); Kannan Essakimuthu MUTHAIYYAN, Gandhinagar, Gujarat (IN); Ritu Nitin LADDHA, Gandhinagar, Gujarat (IN); Santosh Devidas DIWAKAR, Gandhinagar, Gujarat (IN); Mukeshkumar Hirabhai UKAWALA, Gandhinagar, Gujarat (IN); Chintan Sureshbhai DHOLAKIA, Gandhinagar, Gujarat (IN); Jitendrakumar Dashrathial PATEL, Gandhinagar, Gujarat (IN); Sumer Singh CHUNDAWAT, Gandhinagar, Gujarat (IN)

(21) Appl. No.: 18/688,677

(22) PCT Filed: Sep. 19, 2022
(86) PCT No.: PCT/IB22/58830

§ 371 Date: Mar. 1, 2024

(30) Foreign Application Priority Data
Sep. 20, 2021 (IN) 202121042533

(51) Int. Cl. A61K 9/20; A61K 31/4704; C07D 215/60

(52) U.S. Cl. CPC A61K 9/2054; A61K 9/2018; A61K 31/4704; C07D 215/60

(57) ABSTRACT

This invention relates to a field of pharmaceuticals, in particular to desidustat particles having specific particle size distribution and pharmaceutical compositions thereof.

### RELATED APPLICATION

This application claims priority to Indian Provisional Patent Application No. 202121042533, filed on Sep. 20, 2021, the contents of which are incorporated herein in its entirety by reference.

### FIELD OF THE INVENTION

This invention relates to a field of pharmaceuticals, in particular to desidustat particles having specific particle size distribution and pharmaceutical compositions thereof.

### BACKGROUND OF THE INVENTION

International (PCT) Publication No. WO 2014/102818 A1 discloses a series of quinolone compounds that are indicated to have inhibitory activity against Hypoxia-inducible factor (HIF) hydroxylases and to be useful in the treatment of conditions mediated by HIF prolyl hydroxylase including anemia. 2-(1-(Cyclopropylmethoxy)-4-hydroxy-2-oxo-1,2-dihydroquinoline-3-carboxamido) acetic acid (also referred to as “Desidustat”) is one of the compounds disclosed in International (PCT) Publication No. WO 2014/102818 A1. Desidustat has the chemical structure shown as a compound of Formula (I) below:

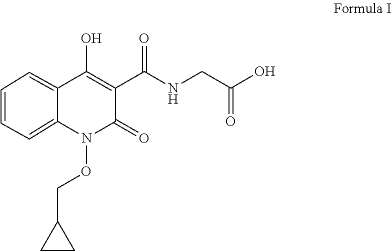 [SMILES: O=C(O)CNC(=O)c1c(O)c2ccccc2n(OCC2CC2)c1=O]

U.S. PG-Publication No. 2019/0359574 A1 discloses process for preparation of quinolone compounds including the compound of Formula (I) and a crystalline form thereof.

International (PCT) Publication No. WO 2021/181360 A1 discloses various pharmaceutical salts of desidustat like calcium, sodium, potassium, lithium, barium, strontium, magnesium, cesium, copper, cobalt, iron, manganese, lead, aluminum, cadmium, silver, zinc, ammonium etc. and pharmaceutical compositions thereof.

Solid oral pharmaceutical dosage forms are popular and useful forms of medications for dispensing pharmaceutically active compounds. A variety of such forms are known, including tablets, and capsules. However, the formulation of an acceptable solid oral pharmaceutical dosage form on a commercial scale is not always straightforward. The formula and process of manufacture must be such as to provide an integral solid dosage form that maintains its integrity until used. The solid dosage form must also possess acceptable dissolution and disintegration properties so as to provide the desired drug release profile in use for the patients.

The present invention provides desidustat particles having specific particle size distribution as disclosed herein, that can be used in pharmaceutical composition to have better dissolution and ultimately better bioavailability.

### SUMMARY OF THE INVENTION

In one general aspect, the present invention provides desidustat particles, wherein at least 90% of the desidustat particles are less than about 250 μm.

In another general aspect, the present invention provides desidustat particles, wherein,
at least 90% of the desidustat particles are less than about 175 μm,
at least 50% of the desidustat particles are less than about 75 μm, and
at least 10% of the desidustat particles are less than about 10 μm.

In another general aspect, the present invention provides desidustat particles having specific surface area value of at least 0.75 m²/g.

In another general aspect, the present invention provides a pharmaceutical composition comprising desidustat particles and at least one pharmaceutically acceptable excipient, wherein at least 90% of the desidustat particles are less than about 250 μm.

In another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition provides a serum or plasma profile for desidustat comprising a mean Cₘₐₓ of from about 1,500 ng/ml to about 15,000 ng/ml, when administered to the patient in need thereof.

In another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition provides a serum or plasma profile for desidustat comprising a mean AUCₗₐₛₜ of from about 10,000 hr*ng/ml to about 75,000 hr*ng/ml, when administered to the patient in need thereof.

In another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition provides a serum or plasma profile for desidustat comprising a mean Tₘₐₓ of from about 0.5 hr to about 7.0 hr, when administered to the patient in need thereof.)

In another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition does not contain (1-(but-3-en-1-yloxy)-4-hydroxy-2-oxo-1,2-dihydroquinoline-3-carbonyl) glycine more than 0.5% by weight of desidustat, as measured by HPLC, after storage for 3 months at 40°±2° C. and 75±5% relative humidity.

In another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition retains at least 95% of the desidustat after storage for 3 months at 40°±2° C. and 75±5% relative humidity.

In another general aspect, the present invention provides a pharmaceutical composition comprising desidustat particles and at least one pharmaceutically acceptable excipient, wherein
at least 90% of the desidustat particles are less than about 175 μm,
at least 50% of the desidustat particles are less than about 75 μm and
at least 10% of the desidustat particles are less than about 10 μm.

In another general aspect, the present invention provides a pharmaceutical composition comprising desidustat particles, wherein at least 90% of the desidustat particles are less than about 250 μm and wherein at least 90% of the desidustat is dissolved within 30 minutes as measured using a USP Apparatus 2 at a paddle rotation speed of 50 rpm in 900 mL of a dissolution medium containing pH 6.8 phosphate buffer at 37° C.±0.5° C.

In another general aspect, the present invention provides a method of treatment for anemia in a patient comprising administering to a patient in need thereof the pharmaceutical composition of the present invention.

### BRIEF DESCRIPTION OF DRAWING

FIG. 1. Powder X-ray diffraction pattern of crystalline desidustat as prepared in example 1.

### DETAILED DESCRIPTION OF THE INVENTION

The inventors of the application have found that desidustat particles with specific particle size distribution are very important to provide better dissolution and ultimately better bioavailability to the pharmaceutical composition.

The term “particles” as used herein refers to individual drug substance particles whether the particles exist singly or are agglomerated.

The phrase “particles are less than” refers to the particle size of the desidustat particles less than the value as mentioned. The particle size is determined on a particle volume basis by using techniques known for the measurement of particle size. Particle size can be characterized by one or more values such as D₉₀, D₅₀ or D₁₀.

The term “D₉₀” as used herein, describes the value of particle size at which 90% of the total volume of particles is comprised of particles of the indicated size.

The term “D₅₀” as used herein, describes the value of particle size at which 50% of the total volume of particles is comprised of particles of the indicated size.

The term “D₁₀” as used herein, describes the value of particle size at which 10% of the total volume of particles is comprised of particles of the indicated size.

The term “about” as used herein is intended to mean approximately, in the region of, roughly, or around. When the term “about” is used in conjunction with a numerical range, it modifies that range by extending the boundaries above and below the numerical values set forth. In general, the term “about” may be used herein to modify a numerical value above and below the stated value by a variance of, for example, 10%.

The term “an effective amount” as used herein means an amount of active compound or pharmaceutical agent that elicits the biological or medicinal response in a tissue, system, animal or human that is being sought by a researcher, veterinarian, medical doctor or other clinician, which includes alleviation or palliation of the symptoms of the disease being treated.

The term “Cₘₐₓ” as used herein refers to maximum plasma concentration achieved after administration of the composition.

The term “AUCₜ” as used herein refers to area under the concentration-time curve from time zero to time t.

The term “AUCₗₐₛₜ” as used herein refers to area under the concentration-time curve from time zero to up to the last measurable concentration.

The term “Tₘₐₓ” as used herein refers to time to maximum plasma concentration.

The term “pharmaceutically acceptable” as used herein indicates that the material does not have properties that would cause one of skill in the art to avoid administration of the material to a patient, taking into consideration the disease or conditions to be treated and the respective route of administration. Further, the material is considered to be safe for administration in humans or animals.

The term “Patient” includes both human and animals. “Mammal” means humans and other mammalian animals.

The term “excipient(s)” or “pharmaceutically acceptable excipient(s)” refers to pharmacologically inactive substances that are added to a pharmaceutical preparation in addition to the active pharmaceutical ingredient. The pharmaceutically acceptable excipients may include one or more diluents, binders, disintegrants, lubricants, glidants, sweeteners/taste masking agents, colorants, flavours, film-forming agents, plasticizers and the like.

The term “method of treatment” as used herein, means any treatment of a disease or disorder in a mammal, including: preventing or protecting against the disease or disorder, that is, causing the clinical symptoms not to develop; inhibiting the disease or disorder, that is, arresting or suppressing the development of clinical symptoms; and/or relieving the disease or disorder, that is, causing the regression of clinical symptoms.

The term “substantially free from any other crystalline form” as used herein refers to the said crystalline Form comprises less than about 1%, more preferably less than about 0.5%, most preferably less than about 0.1% of the other crystalline form. In particular, the other crystalline form is absent. The content of other crystalline form can be measured by using powder X-ray powder diffraction.

In general, desidustat to be used as the starting material may be prepared by the known methods reported in the prior art, for example, by using the process as disclosed in U.S. PG-Publication No. 2019/0359574 A1 which is incorporated herein as reference.

Surprisingly and unexpectedly, the present inventors of the application found that the pharmaceutical composition comprising desidustat particles, wherein at least 90% of the desidustat particles are less than about 250 μm, lead to consistent improved in-vitro dissolution which can be correlated with the improved bioavailability and therefore improved therapeutic efficacy. Moreover, the pharmaceutical composition of the present invention was found to be stable for more than 3 months when stored at 40°±2° C. and 75±5% relative humidity.

Thus the use of desidustat particles of the present invention in the pharmaceutical composition resulted in improved dissolution profile of desidustat. An improved dissolution profile has significant advantages including the improvement of bioavailability of the desidustat in-vivo. In some embodiments, the improved dissolution profile is observed in-vitro. Alternatively, the improved dissolution profile is observed in-vivo by the observation of an improved bioavailability profile. Standard methods for determining the dissolution profile of a material in-vitro are available in the art. A suitable method to determine an improved dissolution profile in-vitro may include determining the concentration of the sample material in a solution over a period of time.

In one general aspect, the present invention provides desidustat particles, wherein at least 90% of the desidustat particles are less than about 250 μm.

In one embodiment of the above aspect, the present invention provides desidustat particles, wherein at least 90% of the desidustat particles are less than about 250 μm and at least 50% of the desidustat particles are greater than about 5 μm.

In another embodiment, the present invention provides desidustat particles, wherein at least 90% of the desidustat particles are less than about 200 μm.

In another embodiment, the present invention provides desidustat particles, wherein at least 90% of the desidustat particles are less than about 175 μm.

In another embodiment, the present invention provides desidustat particles, wherein at least 90% of the desidustat particles are less than about 200 μm, for example, less than about 175 μm, less than about 150 μm, less than about 130 μm, less than about 110 μm or less than about 100 μm.

In another embodiment, the present invention provides desidustat particles, wherein at least 90% of the desidustat particles are in the range of from about 50 μm to about 175 μm.

In another embodiment, the present invention provides desidustat particles, wherein at least 50% of the desidustat particles are less than about 150 μm.

In another embodiment, the present invention provides desidustat particles, wherein at least 50% of the desidustat particles are less than about 75 μm.

In another embodiment, the present invention provides desidustat particles, wherein at least 50% of the desidustat particles are less than about 75 μm, for example, less than about 50 μm, less than about 40 μm, less than about 30 μm, less than about 20 μm or less than about 10 μm.

In another embodiment, the present invention provides desidustat particles, wherein at least 10% of the desidustat particles are less than about 30 μm.

In another embodiment, the present invention provides desidustat particles, wherein at least 10% of the desidustat particles are less than about 10 μm.

In another embodiment, the present invention provides desidustat particles, wherein at least 10% of the desidustat particles are less than about 20 μm, for example, less than about 15 μm, less than about 10 μm, less than about 5 μm or less than about 3 μm.

In another embodiment, the present invention provides desidustat particles having specific surface area value of at least 0.75 m²/g.

In another embodiment, the present invention provides desidustat particles having specific surface area value of at least 1.0 m²/g.

In another embodiment, the present invention provides desidustat particles, wherein the particles have specific surface area value in the range of from about 0.9 m²/g to about 4.0 m²/g.

In another embodiment, the present invention provides desidustat particles, wherein the particles have specific surface area value in the range of from about 1.0 m²/g to about 3.0 m²/g.

In another embodiment, the desidustat particles of the present invention are present in a pharmaceutical composition.

In another embodiment, the desidustat particles of the present invention are crystalline in nature.

In another embodiment, the crystalline desidustat particles are characterized by at least three powder X-ray diffraction pattern peaks expressed in degree 2θ selected from 8.0°, 8.9°, 10.6°, 11.3°, 16.1°, 25.5°, and 26.4°+0.2°.

In another embodiment, the crystalline desidustat particles are characterized by powder X-ray diffraction pattern peaks expressed in degree 2θ at 8.0°, 8.9°, 10.6°, 11.3°, 16.1°, 25.5°, and 26.4°=0.2°.

In another embodiment, the crystalline desidustat particles are characterized by powder X-ray diffraction pattern peaks expressed in degree 2θ at 8.0°, 8.9°, 11.3° and 25.5°±0.2°.

In another embodiment, the crystalline desidustat particles are characterized by at least three powder X-ray diffraction pattern peaks expressed in degree 2θ selected from 8.0°, 8.9°, 10.6°, 11.3°, 16.1°, 25.5°, and 26.4°±0.2° and are substantially free from any other crystalline form.

In another embodiment, the crystalline desidustat particles are characterized by powder X-ray diffraction pattern peaks expressed in degree 2θ at 8.0°, 8.9°, 10.6°, 11.3°, 16.1°, 25.5°, and 26.4°±0.2°, are substantially free from any other crystalline form.

In another embodiment, the crystalline desidustat particles are characterized by powder X-ray diffraction pattern peaks expressed in degree 2θ at 8.0°, 8.9°, 11.3° and 25.5°±0.2°, and are substantially free from any other crystalline form.

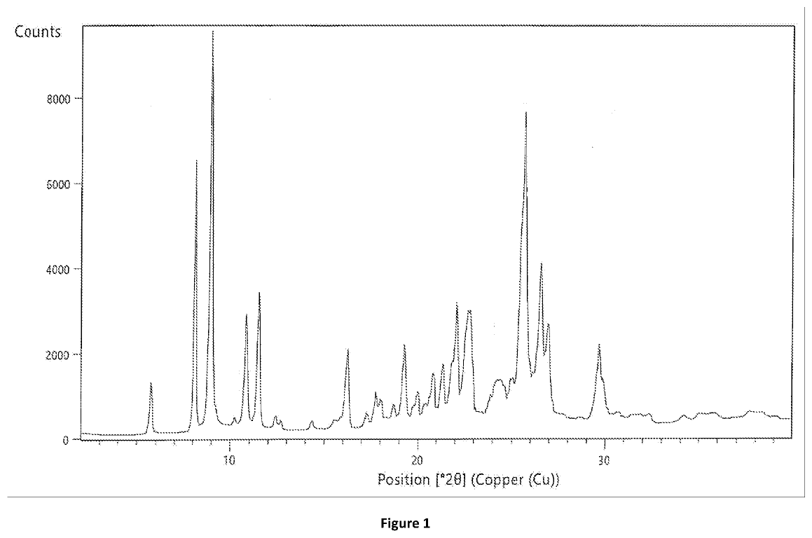

In another embodiment, the crystalline desidustat particles are characterized by powder X-ray diffraction pattern substantially as same as depicted in FIG. 1.

In another general aspect, the present invention provides desidustat particles, wherein,
at least 90% of the desidustat particles are less than about 175 μm,
at least 50% of the desidustat particles are less than about 75 μm and
at least 10% of the desidustat particles are less than about 10 μm.

In another general aspect, the present invention provides desidustat particles, wherein at least 90% of the desidustat particles are less than about 250 μm, wherein the desidustat particles are crystalline, and wherein the crystalline desidustat particles are characterized by at least three powder X-ray diffraction pattern peaks expressed in degree 2θ selected from 8.0°, 8.9°, 10.6°, 11.3°, 16.1°, 25.5°, and 26.4°±0.2°.

The desidustat particles of the present invention can be prepared by milling the desidustat using techniques known for the reduction of particle size, for example, multi-milling, jet milling, ball milling, or similar other techniques known in the art.

Thus in one embodiment, the desidustat particles are prepared by a process of milling.

In another embodiment, the desidustat particles are obtained by dry or wet milling.

In another general aspect, the present invention provides a pharmaceutical composition comprising desidustat particles and a pharmaceutically acceptable excipient, wherein at least 90% of the desidustat particles are less than about 250 μm.

In one embodiment of the above aspect, the present invention provides a pharmaceutical composition comprising desidustat particles and at least one pharmaceutically acceptable excipient, wherein at least 90% of the desidustat particles are less than about 250 μm and at least 50% of the desidustat particles are greater than about 5 μm.

In another embodiment of the above aspect, the present invention provides a pharmaceutical composition comprising desidustat particles and at least one pharmaceutically acceptable excipient, wherein at least 75% of the desidustat is dissolved within 30 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 80% of the desidustat is dissolved within 30 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 90% of the desidustat is dissolved within 30 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 70% of the desidustat is dissolved within 10 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 75% of the desidustat is dissolved within 15 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 85% of the desidustat is dissolved within 15 minutes as measured using a USP Apparatus 2 at a paddle rotation speed of 50 rpm in 900 mL of a dissolution medium containing pH 6.8 phosphate buffer at 37° C.±0.5° C.

In another embodiment of the above aspect, the present invention provides a pharmaceutical composition comprising desidustat particles and at least one pharmaceutically acceptable excipient, wherein at least 70% of the desidustat is dissolved within 10 minutes and at least 80% of the desidustat is dissolved within 30 minutes, as measured using a USP Apparatus 2 at a paddle rotation speed of 50 rpm in 900 mL of a dissolution medium containing pH 6.8 phosphate buffer at 37° C.±0.5° C.

In another embodiment, at least 90% of the desidustat particles in the pharmaceutical composition of the present invention are less than about 200 μm.

In yet another embodiment, at least 90% of the desidustat particles in the pharmaceutical composition of the present invention are less than about 175 μm.

In yet another embodiment, at least 90% of the desidustat particles in the pharmaceutical composition of the present invention are in the range of from about 50 μm to about 175 μm.

In still yet another embodiment, the present invention provides a pharmaceutical composition comprising desidustat particles and at least one pharmaceutically acceptable excipient, wherein at least 90% of the desidustat particles are less than about 200 μm, for example, less than about 175 μm, less than about 150 μm, less than about 130 μm, less than about 110 μm or less than about 100 μm.

In another embodiment, the present invention provides a pharmaceutical composition comprising desidustat particles and at least one pharmaceutically acceptable excipient, wherein at least 50% of the desidustat particles are less than about 150 μm.

In another embodiment, at least 50% of the desidustat particles in the pharmaceutical composition of the present invention are less than about 75 μm.

In yet another embodiment, at least 50% of the desidustat particles in the pharmaceutical composition of the present invention are less than about 75 μm, for example, less than about 50 μm, less than about 40 μm, less than about 30 μm, less than about 20 μm or less than about 10 μm.

In another embodiment, the present invention provides a pharmaceutical composition comprising desidustat particles and at least one pharmaceutically acceptable excipient, wherein at least 10% of the desidustat particles are less than about 30 μm.

In yet another embodiment, at least 10% of the desidustat particles in the pharmaceutical composition of the present invention are less than about 10 μm.

In still yet another embodiment, at least 10% of the desidustat particles in the pharmaceutical composition of the present invention are less than about 20 μm, for example, less than about 15 μm, less than about 10 μm, less than about 5 μm or less than about 3 μm.

In another embodiment, the pharmaceutical composition of the present invention comprises from about 25 mg to about 150 mg of desidustat, for example, 50 mg, 100 mg, 125 mg.

In another embodiment, the desidustat particles in the pharmaceutical composition of the present invention are crystalline.

In yet another embodiment, the crystalline desidustat particles in the pharmaceutical composition are characterized by at least three powder X-ray diffraction pattern peaks expressed in degree 20 selected from 8.0°, 8.9°, 10.6°, 11.3°, 16.1°, 25.5°, and 26.4°±0.2°.

In yet another embodiment, the crystalline desidustat particles in the pharmaceutical composition are characterized by powder X-ray diffraction pattern peaks expressed in degree 20 at 8.0°, 8.9°, 11.3° and 25.5°±0.2°.

In still yet another embodiment, the crystalline desidustat particles in the pharmaceutical composition are characterized by powder X-ray diffraction pattern peaks expressed in degree 2θ at 8.0°, 8.9°, 10.6°, 11.3°, 16.1°, 25.5°, and 26.4°±0.2°.

In still yet another embodiment, the present invention provides a pharmaceutical composition comprising desidustat particles and at least one pharmaceutically acceptable excipient, wherein at least 90% of the desidustat particles are less than about 250 μm, wherein the desidustat particles are crystalline, and wherein the crystalline desidustat particles are characterized by at least three powder X-ray diffraction pattern peaks expressed in degree 2θ selected from 8.0°, 8.9°, 10.6°, 11.3°, 16.1°, 25.5°, and 26.4°±0.2°.

The pharmaceutically acceptable excipients for use in the pharmaceutical composition of the present invention may comprise one or more diluents, binders, disintegrants, lubricants, glidants, sweeteners/taste masking agents, flavours, film-forming agents, plasticizers etc.

Diluent refers to an ingredient (excipient) in a pharmaceutical composition that lacks pharmacological activity but may be pharmaceutically necessary or desirable, e.g. to enhance or improve the properties of the pharmaceutical blend for manufacturing or physiological purposes. Non-limiting examples of diluent(s) include starch and its processed and co-processed derivatives, saccharides, disaccharides, sucrose, lactose, polysaccharides, cellulose, cellulose ethers, cellulose acetate, hydroxypropylcellulose, sugar alcohols, xylitol, sorbitol, maltitol, lactitol, microcrystalline cellulose, magnesium or calcium or sodium carbonate, lactose, lactose monohydrate, di-calcium phosphate, compressible sugars, di-basic calcium phosphate dihydrate, mannitol, lactose anhydrous, magnesium oxide, maltodextrin, maltose, pullulan, sodium alginate, sodium bicarbonate, calcium silicate, calcium sulphate and tribasic calcium phosphate or a suitable combination thereof.

Binder refers to any pharmaceutically acceptable substance which can be used to bind the active and inert components together to maintain cohesive and discrete portions. Non-limiting examples of binder(s) include chitosan, hydrogenated castor oil, sodium alginate, carbomers, cellulose acetate phthalate, povidone, sugar, hydroxypropyl methylcellulose, hydroxypropylcellulose, starch, alginic acid, pregelatinized starch, acacia, tragacanth, ethylcellulose, acrylic and methacrylic acid co-polymers or a suitable combination thereof.

Disintegrant or disintegrating agent refers to a substance which, upon addition to a solid preparation, facilitates its break-up or disintegration after administration and permits the release of an active ingredient as efficiently as possible to allow for its rapid dissolution. Non-limiting examples of disintegrant(s) include maize starch, sodium starch glycolate, croscarmellose sodium, crospovidone, microcrystalline cellulose, sodium carboxymethyl starch, pregelatinized starch, agar, carboxymethyl cellulose calcium or sodium, low-substituted hydroxypropyl cellulose, magnesium aluminium silicate, methyl cellulose, polacrilin potassium, and alginic acid or a suitable combination thereof.

Lubricant refers to an excipient, which is added to a powder blend to prevent the compacted powder mass from sticking to the equipment during the tableting or encapsulation process. It aids the ejection of the tablet form the dies, and can improve powder flow. Non-limiting examples of lubricant(s) include magnesium stearate, stearic acid, silica, fats, zinc or sucrose or sodium or calcium stearate, castor oil, hydrogenated castor oil, polyethylene glycol and its derivatives, sodium stearyl fumarate, talc, or fatty acids including lauric acid, oleic acid, glyceryl behenate, glyceryl monostearate, and C₁-C₁₀ fatty acid or a suitable combination thereof.

Glidant refers to an excipient, which is used in tablet and capsule formulations to improve flow-properties during tablet compression and to produce an anti-caking effect. Non-limiting examples of glidants(s) include colloidal silicon dioxide, talc, fumed silica, starch, starch derivatives, and bentonite or a suitable combination thereof.

Suitable taste masking agents may include one or more of polymers, sweeteners and flavours. The polymers may include one or more of cellulose acetate, polymethacrylates, hydroxypropyl methylcellulose, hydroxypropylcellulose, hydroxylethylcellulose; and the like. Suitable sweeteners that may be used, comprises saccharides such as sucrose, dextrose, glucose, maltose, dextrins, D-tagatose, trehalose, dried invert sugar, fructose, levulose, galactose, corn syrup solids, and the like, alone or in combination. Other examples of sweeteners comprise sodium saccharin; aspartame; sugarless sweeteners including polyhydric alcohols such as sorbitol, mannitol, xylitol, glycerol, hydrogenated starch hydrolysates, maltitol, isomaltitol, erythritol, lactitol and the like, alone or in combination. Suitable flavours that may be used, comprise cinnamon, wintergreen, eucalyptus, spearmint, peppermint, menthol, anise as well as fruit flavours such as apple, pear, peach, strawberry, cherry, apricot, orange, watermelon, banana and the like; bean-derived flavours, such as coffee, cocoa and the like or mixtures thereof.

The pharmaceutical composition of desidustat may be developed in the form of tablets, capsules, powders, pellets, granules, microspheres, minitablets or any suitable solid unit forms known to person skilled in the art; mouth dissolving tablets; dispersible tablets; effervescent tablets; trilayer tablets; inlay tablets. The preferred dosage forms are tablets and capsules filled with pellets, granules or minitablets as these are more convenient and easier to administer.

In one embodiment, the pharmaceutical composition comprising the desidustat particles of the present invention is in the form of tablet. The tablet may be in the form of coated or uncoated tablet. In another embodiment, the pharmaceutical composition is in the form of capsule.

The pharmaceutical composition of desidustat may be manufactured by using various techniques known to the person skilled in the art, such as, but not limited to direct compression, wet granulation, dry granulation, hot melt granulation, hot melt extrusion, fluidized bed granulation, extrusion, and solvent evaporation.

The components of the pharmaceutical composition defined hereinbefore can be brought together into a suitable composition for oral administration according to standard practice and procedures well known in the art of pharmaceutical science using conventional formulation and manufacturing techniques.

In an embodiment, desidustat composition may be prepared by granulating the admixture of desidustat and one or more pharmaceutical excipients. The resulting granules may be compressed to form tablets or filled in hard gelatin capsules.

In another embodiment, a stable desidustat composition may be developed in the form of pellets, which may be prepared by coating one or more layers of desidustat on non-pareil sugar seeds or inert cores. The resulting pellets may be admixed with pharmaceutical excipients and filled into hard gelatin capsules or may be compressed with pharmaceutical excipients to form tablets.

The composition may be seal coated and finally film coated. The composition may be coated with ready color mix systems (such as opadry color mix systems).

In yet another embodiment, the pharmaceutical composition may involve one or more manufacturing process to obtain a single unitary dosage form i.e., wherein the drug is processed by granulation techniques as discussed above and finally compacted to yield a single dosage form.

In yet another embodiment, the stable pharmaceutical composition may be prepared by a process, wherein the process comprises the steps of:
(a) preparing a homogeneous melt by mixing desidustat with a suitable binder;
(b) cooling the melt of step (a), milling and sizing to obtain granules;
(c) blending the granules of step (b) with one or more pharmaceutically acceptable excipients;
(d) compressing the blend of step (c) to obtain tablets; and
(e) optionally, coating the tablets.

In yet another embodiment, the stable pharmaceutical composition may be prepared by a process, wherein the process comprises the steps of:
(a) mixing desidustat, and one or more pharmaceutically acceptable excipients;
(b) granulating the mixture of step (a) with or without a binder solution;
(c) blending granules obtained in step (b) with one or more pharmaceutically acceptable excipients; and
(d) compressing the blended granules into a tablet.

In yet another embodiment, the stable pharmaceutical composition may be prepared by a process, wherein the process comprises the steps of:
(a) preparing solution or dispersion of desidustat, and one or more pharmaceutically acceptable excipients;
(b) spraying the solution or dispersion of desidustat on inert cores;
(c) blending coated cores obtained in step (b) with one or more pharmaceutically acceptable excipients; and
(d) compressing the blended granules into a tablet.

The present compositions may, if desired, be presented in a pack or dispenser device containing one or more unit dosage forms containing the active ingredient. Such a pack or device may, for example, comprise metal or plastic foil, such as a blister pack. For example, the present composition may be packed in Alu/Alu blister or PVC-PVDC pack.

In another embodiment, a pharmaceutical composition comprises:
(i) from 30 to 45% by weight of desidustat particles, wherein at least 90% of the desidustat particles are less than about 250 μm;
(ii) from 30 to 55% by weight of a diluent selected from microcrystalline cellulose, lactose, starch or mannitol;
(iii) from 3 to 7% by weight of a disintegrant selected from croscarmellose sodium, crospovidone, pregelatinized starch, sodium starch glycolate or low-substituted hydroxypropyl cellulose;
(iv) from 2 to 6% by weight of a binder selected from hydroxypropyl methylcellulose, polyvinylpyrrolidone or hydroxypropyl cellulose;
(v) from 0.5 to 3% by weight of a glidant;
(vi) from 0.5 to 3% by weight of a lubricant; and
(vii) an optional coating.

In yet another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition does not contain (1-(but-3-en-1-yloxy)-4-hydroxy-2-oxo-1,2-dihydroquinoline-3-carbonyl) glycine more than 0.5% by weight of desidustat, as measured by HPLC, after storage for 3 months at 40°±2° C. and 75±5% relative humidity.

In yet another general aspect, the present invention provides a pharmaceutical composition comprising a therapeutically effective amount of desidustat particles and at least one pharmaceutically acceptable excipient, wherein at least 90% of the desidustat particles are less than about 250 μm, and wherein the composition retains at least 95% of the desidustat after storage for 3 months at 40°±2° C. and 75±5% relative humidity.

In another general aspect, the present invention provides a method of treatment of anemia in a patient, which comprises administering to a patient in need thereof a pharmaceutical composition comprising desidustat particles, wherein at least 90% of the desidustat particles are less than about 250 μm and wherein the desidustat is administered in the range of 1 mg to 500 mg, for example, in the range of 5 mg to 400 mg, in the range of 10 mg to 300 mg.

In one embodiment of the above general aspect, a pharmaceutical composition provides a serum or plasma profile for desidustat comprising a mean Cₘₐₓ of from about 1,500 ng/ml to about 15,000 ng/ml, when administered to the patient in need thereof.

In another embodiment of the above general aspect, a pharmaceutical composition provides a serum or plasma profile for desidustat comprising a mean AUCₗₐₛₜ of from about 10,000 hr*ng/ml to about 75,000 hr*ng/ml, when administered to the patient in need thereof.

In yet another embodiment of the above general aspect, a pharmaceutical composition provides a serum or plasma profile for desidustat comprising a mean Tₘₐₓ of from about 0.5 hr to about 7.0 hr, when administered to the patient in need thereof.

In yet another embodiment of the above general aspect, the pharmaceutical composition has a mean fed/fasted ratio of the area under the plasma concentration (AUC) versus time curve of from about 0.60 to about 0.90 and a mean fed/fasted ratio of the maximum plasma concentration (Cₘₐₓ) from about 0.40 to about 0.70.

The particle size of desidustat may be determined by using techniques known for the measurement of particle size, for example by Malvern light scattering, a laser light scattering technique. Alternatively, the particle size of desidustat in the pharmaceutical composition can be measured by hot stage microscopy.

Hot stage microscopy technique is a fusion of micromeritics and analysis at higher temperatures. The Formulation is placed on the graduated sample loaded in which the particle size can be determined using those graduations. The heat is applied and as the furnace heatens, the materials start to melt over near to its melting range. This causes the change in the structures and the morphology of the structure also changes and all the changes in the structure and the size of the particle is captured by hot stage microscopy.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Analytical Methods

### (i) Particle Size and Specific Surface Area Analysis Method:

Particle size was determined by Malvern Mastersizer 3000 with Hydro MV accessory, laser diffraction particle size analyser. About 100 mg of drug substance weighed and transferred into 100 mL of glass beaker. About 10-15 drops of dispersant sunflower oil:cyclohexane (80:20) were added and the lumps were broken with glass rod for about 3 to 5 min. 20 mL of dispersant was added into the same glass beaker and the content were shaken for 3-5 minutes with glass rod to mix well. Background measurement using dispersant was performed. When the blank correction/background measurement was over, sample slurry (with continuous manual shaking) was added in the sampler at 1500 rpm to get the target obscuration value between 10% and 30% and remained constant. Then the histogram was recorded.

### EXAMPLES

### Example—1

### Preparation of Desidustat

Ethyl (1-(cyclopropylmethoxy)-4-hydroxy-2-oxo-1,2-dihydroquinoline-3-carbonyl) glycinate (21 Kg, 58 mmol), methanol (147 L) and water (84 L) were added in a reactor at 25-35° C. Sodium hydroxide (5.83 Kg, 145 mmol) in water (61 L) was then charged and the resultant reaction mass was stirred at 30-40° C. for 2 hours. Water (588 L) was charged at 25-40° C. to the reaction mixture. 15% hydrochloric solution (42 L) was added to the reaction mixture and stirred for 60-70 minutes. The reaction mass was filtered and dried at 25-35° C. for six hours and 50-60° C. for four hours followed by multi-milling using 5 mm sieve and then dried at 60-70° C. for four hours to obtain desidustat.

Particle size: D₉₀=366 μm.

The above material was further subjected to multi-milling using 0.2 mm sieve to obtain 18.73 kg (96.9%) of desidustat.

Powder X-ray diffraction pattern is as set forth in FIG. 1.

Particle size distribution: D₁₀=0.80 μm, D₅₀=22.7 μm, D₉₀=130 μm.

Specific surface area=2 m²/g.

### Example-2

| | | Quantity |
|---|---|---|
| Sr. No. | Name of ingredient | (% w/w) |
| 1 | Desidustat | 38.83 |
| 2 | Microcrystalline cellulose | 42.72 |
| 3 | Croscarmellose sodium | 4.85 |
| 4 | Lactose monohydrate | 4.85 |
| 5 | Hypromellose 3 CPS | 3.88 |
| 6 | Talc | 0.97 |
| 7 | Magnesium stearate | 0.97 |
| 8 | Opadry pink | 2.91 |

### Manufacturing Process:

1. Desidustat, microcrystalline cellulose, lactose monohydrate, part quantity of croscarmellose sodium were sifted through an appropriate sized sieve and mixed together.
2. Hypromellose was dissolved in purified water to obtain binder solution.
3. The powder mix of step (1) was granulated with the help of the binder solution, prepared in step (2).
4. The granules were dried, sized and mixed with remaining quantity of croscarmellose sodium and then lubricated with talc and magnesium stearate.
5. The lubricated blend was compressed into tablets using tablet compression machine.
6. The tablets were film coated with opadry solution.

### Example-3

| | | Quantity |
|---|---|---|
| Sr. No. | Name of ingredient | (% w/w) |
| 1 | Desidustat | 40.00 |
| 2 | Microcrystalline cellulose | 44.00 |
| 3 | Croscarmellose sodium | 5.00 |
| 4 | Lactose monohydrate | 5.00 |
| 5 | Hypromellose | 4.00 |
| 6 | Talc | 1.00 |
| 7 | Magnesium stearate | 1.00 |
| 8 | Purified Water | q.s. |

### Manufacturing Process:

1. Desidustat, microcrystalline cellulose, lactose monohydrate, part quantity of croscarmellose sodium were sifted through an appropriate sized sieve and mixed together.
2. Hypromellose was dissolved in purified water to obtain binder solution.
3. The powder mix of step (1) was granulated with the help of the binder solution, prepared in step (2).
4. The granules were dried, sized and mixed with remaining quantity of croscarmellose sodium and then lubricated with talc and magnesium stearate.
5. The lubricated blend was compressed into tablets using tablet compression machine.

Particle size of desidustat used in the composition

| | D₁₀ (μm) | D₅₀ (μm) | D₉₀ (μm) |
|---|---|---|---|
| Unmilled desidustat | 2.16 | 98.3 | 366 |
| Milled desidustat | 0.80 | 22.7 | 130.0 |

Dissolution data of Example 3:

Dissolution media: pH 6.8 phosphate buffer, 900 ml, paddle, 50 rpm
(USP Apparatus 2) at 37° C. ± 0.5° C.

| Time | (Unmilled desidustat) | | (Milled desidustat) | |
|---|---|---|---|---|
| (min.) | % Dissolution | % RSD | % Dissolution | % RSD |
| 10 | 58.7 | 8.4 | 72.49 | 9.5 |
| 15 | 73.2 | 6.2 | 86.72 | 7.2 |
| 30 | 82.0 | 4.2 | 92.50 | 6.8 |

### Examples—4, 5, 6, and 7

Similarly, by taking desidustat with different particle size, the tablets having composition as per example 3 were prepared.

The composition of examples 4, 5, 6, and 7 are as shown below:

| | | Quantity |
|---|---|---|
| Sr. No. | Name of ingredient | (% w/w) |
| 1 | Desidustat | 40.00 |
| 2 | Microcrystalline cellulose | 44.00 |
| 3 | Croscarmellose sodium | 5.00 |
| 4 | Lactose monohydrate | 5.00 |
| 5 | Hypromellose | 4.00 |
| 6 | Talc | 1.00 |
| 7 | Magnesium stearate | 1.00 |
| 8 | Purified Water | q.s. |

The particle size of desidustat used in the composition of examples 4, 5, 6, 7 and 8 are as shown below:

| | | | | Specific surface |
|---|---|---|---|---|
| | Particle size of desidustat | | | area of desidustat |
| Examples | D₁₀ (μm) | D₅₀ (μm) | D₉₀ (μm) | particles (m²/g) |
| Example 4 | 1.42 | 45.5 | 186 | 0.98 |
| Example 5 | 1.07 | 6.48 | 100 | 1.82 |
| Example 6 | 0.900 | 2.35 | 39.5 | 3.17 |
| Example 7 | 0.922 | 3.01 | 28.6 | 2.83 |

Dissolution data of Example 4, 5, 6, 7 and 8:

Dissolution media: pH 6.8 phosphate buffer, 900 ml, paddle, 50 rpm
(USP Apparatus 2) at 37° C. ± 0.5° C.

| | Unmilled desidustat | Milled desidustat | | | |
|---|---|---|---|---|---|
| Time | % Dissolution | % Dissolution (% RSD) | | | |
| (min.) | (% RSD) | Example 4 | Example 5 | Example 6 | Example 7 |
| 10 | 58.7 (8.4) | 84.4 (8.6) | 79.5 (6.3) | 78.5 (13.3) | 71.3 (9.0) |
| 15 | 73.2 (6.2) | 90.8 (7.1) | 87.1 (3.8) | 91.1 (6.8) | 90.2 (4.7) |
| 20 | — | 94.8 (4.9) | 92.3 (2.5) | 94.4 (6.4) | 94.2 (3.3) |
| 30 | 82.0 (4.2) | 96.9 (4.3) | 96.4 (1.9) | 96.5 (5.4) | 98.5 (2.6) |

### Results:

A comparative dissolution study of desidustat immediate release tablets manufactured using unmilled and milled desidustat was studied at pH 6.8 phosphate buffer to understand the impact of particle size on dissolution. A significant difference in dissolution with milled and unmilled desidustat was observed at pH 6.8 phosphate buffer and higher dissolution was observed for desidustat tablets manufactured with milled desidustat. The effect of particle size of desidustat might also have an impact on the in-vivo performance.

Stability data of Example 3:

| | | 40° ± 2° C. & 75 ± 5% RH | | |
|---|---|---|---|---|
| Test | Initial | 1 month | 3 months | 6 months |
| Water content (% w/w) | 3.08 | 2.61 | 2.46 | 2.94 |
| Assay (%) | 100.00 | 98.2 | 99.7 | 98.8 |

Pharmacokinetic data of Example 3:

| PK parameter | Fasting data | Fed data |
|---|---|---|
| [Mean ± SD] | (n = 48) | (n = 48) |
| Cₘₐₓ (ng/ml) | 5495.575 ± 1238.800 | 3247.965 ± 1376.056 |
| Tₘₐₓ (hr) | 1.75 ± 1.04 | 3.721 ± 1.396 |
| AUCₗₐₛₜ (hr*ng/ml) | 33704.988 ± 12908.910 | 25558.683 ± 9250.328 |

While the invention has been described in terms of its specific embodiments, certain modifications and equivalents will be apparent to those skilled in the art and are intended to be included within the scope of the invention.

1-45. (canceled)

46. Desidustat particles, wherein at least 90% of the desidustat particles are less than about 250 μm.

47. The desidustat particles according to claim 46, wherein at least 50% of the desidustat particles are greater than about 5 μm.

48. The desidustast particles according to claim 46, wherein at least 90% of the desidustat particles are less than about 200 μm.

49. The desidustast particles according to claim 47, wherein at least 50% of the desidustat particles are less than about 150 μm.

50. The desidustast particles according to claim 46, wherein at least 10% of the desidustat particles are less than about 30 μm.

51. The desidustat particles according to claim 46, wherein the desidustat particles are crystalline, and the crystalline desidustat particles are characterized by at least three powder X-ray diffraction pattern peaks expressed in degree 2θ selected from 8.0°, 8.9°, 10.6°, 11.3°, 16.1°, 25.5°, and 26.4°+0.2°.

52. Desidustat particles, wherein
at least 90% of the desidustat particles are less than about 175 μm,
at least 50% of the desidustat particles are less than about 75 μm, and
at least 10% of the desidustat particles are less than about 10 μm.

53. Desidustat particles having specific surface area value of at least 0.75 m²/g.

54. A pharmaceutical composition comprising desidustat particles of claim 46 and one or more pharmaceutically acceptable excipients.

55. The pharmaceutical composition according to claim 54, wherein at least 50% of the desidustat particles are greater than about 5 μm.

56. The pharmaceutical composition according to claim 54, wherein at least 90% of the desidustat is dissolved within 30 minutes as measured using a USP Apparatus 2 at a paddle rotation speed of 50 rpm in 900 mL of a dissolution medium containing pH 6.8 phosphate buffer at 37° C.±0.5° C.

57. The pharmaceutical composition according to claim 54, wherein the composition comprises from about 25 mg to about 150 mg of desidustat.

58. The pharmaceutical composition according to claim 54, wherein the composition is a tablet or capsule.

59. A pharmaceutical composition comprising a therapeutically effective amount of desidustat particles and at least one pharmaceutically acceptable excipient, wherein at least 90% of the desidustat particles are less than about 250 μm, and wherein the composition provides a serum or plasma profile for desidustat comprising:
(i) a mean of Cₘₐₓ of from about 1,500 ng/ml to about 15,000 ng/ml, when administered to a patient in need thereof, or
(ii) a mean of AUCₗₐₛₜ of from about 10,000 hr*ng/ml to about 75,000 hr*ng/ml, when administered to a patient in need thereof, or
(iii) a Tₘₐₓ of from about 0.5 hr to about 7.0 hr, when administered to a patient in need thereof.

60. A pharmaceutical composition comprising a therapeutically effective amount of desidustat particles and at least one pharmaceutically acceptable excipient, wherein at least 90% of the desidustat particles are less than about 250 μm, and wherein the composition does not contain 1-(but-3-en-1-yloxy)-4-hydroxy-2-oxo-1,2-dihydroquinoline-3-carbonyl) glycine more than 0.5% by weight of desidustat, as measured by HPLC, after storage for 3 months at 40°±2° C. and 75±5% relative humidity.

61. A pharmaceutical composition comprising a therapeutically effective amount of desidustat particles and at least one pharmaceutically acceptable excipient, wherein at least 90% of the desidustat particles are less than about 250 μm, and wherein the composition retains at least 95% of the desidustat after storage for 3 months at 40°±2° C. and 75±5% relative humidity.

62. A pharmaceutical composition comprising:
(i) from 30 to 45% by weight of desidustat particles, wherein at least 90% of the desidustat particles are less than about 250 μm;
(ii) from 30 to 55% by weight of a diluent selected from microcrystalline cellulose, lactose, starch or mannitol;
(iii) from 3 to 7% by weight of a disintegrant selected from croscarmellose sodium, crospovidone, pregelatinized starch, sodium starch glycolate or low-substituted hydroxypropyl cellulose;
(iv) from 2 to 6% by weight of a binder selected from hydroxypropyl methylcellulose, polyvinylpyrrolidone or hydroxypropylcellulose;
(v) from 0.5 to 3% by weight of a glidant;
(vi) from 0.5 to 3% by weight of a lubricant; and
(vii) an optional coating.

63. A method of treatment for anemia in a patient comprising administering to a patient in need thereof the pharmaceutical composition according to claim 54.

64. A method of treatment for anemia in a patient comprising administering to a patient in need thereof the pharmaceutical composition according to claim 59.

65. A method of treatment for anemia in a patient comprising administering to a patient in need thereof the pharmaceutical composition according to claim 62.
